(19) United States
(12) Patent Application Publication
(10) Pub. No.: US 2025/0339312 A1
(43) Pub. Date: Nov. 6, 2025

(54) DOUBLE BALLOON BREACH DETECTION METHOD AND PREVENTION

(71) Applicant: Medtronic CryoCath, LP, Pointe-Claire (CA)

(72) Inventor: Rahmani Sara, Saint-Laurent (CA)

(21) Appl. No.: 18/854,267

(22) PCT Filed: Apr. 18, 2023
(86) PCT No.: PCT/CA2023/050519

§ 371 Date: Oct. 4, 2024

Related U.S. Application Data
(60) Provisional application No. 63/364,397, filed on May 9, 2022.

(51) Int. Cl. A61F 7/12; A61F 7/00; A61M 1/00; A61M 25/00; A61M 25/10

(52) U.S. Cl. CPC A61F 7/123; A61F 7/0085; A61M 1/74; A61M 25/1011; A61F 2007/0057; A61F 2007/0096; A61F 2007/126; A61M 2025/0002; A61M 2205/15; A61M 2205/18; A61M 2205/3313; A61M 2205/3334; A61M 2205/3368

(57) ABSTRACT

A system includes a console and a balloon catheter with an inner balloon and an outer balloon. The console is fluidly coupled to the balloon catheter via aa first vacuum line and and a second vacuum line. The console includes processing circuitry configured to determine whether at least one breach condition is satisfied based on at least one parameter measured by sensors of the system. Responsive to determining that at least one of the breach conditions is satisfied, the processing circuitry is configured to cause, at a first time, passive suction to be exerted on the first vacuum line and active suction to be exerted on the second vacuum line. Additionally, the processing circuitry is configured to cause, at a second time that is after the first time, active suction to be exerted on first vacuum line and the second vacuum line.

### CROSS-REFERENCE TO RELATED APPLICATION

The present application is related to and claims benefit under 35 U.S.C. § 119(e) from U.S. Provisional Patent Application Ser. No. 63/364,397, filed 9 May 2022, entitled “DOUBLE BALLOON BREACH DETECTION METHOD AND PREVENTION”, the entire contents of which being incorporated herein by reference.

### TECHNICAL FIELD

The present technology is related to ablation catheters. In particular, various examples of the present technology are related to balloon catheters for performing cryogenic techniques.

### BACKGROUND

A cryogenic device, such as a catheter, may employ fluids with low operating temperatures, or cryogens, to selectively freeze, or “cold-treat”, targeted tissues within the body. Such medical devices may be relatively non-invasive and allow for precise treatment of localized discrete tissues that are otherwise inaccessible. For instance, catheters may be easily inserted and navigated through blood vessels and arteries, allowing non-invasive access to areas of the body with relatively little trauma.

In some examples, an ablation system may induce a net transfer of heat flow from target tissue to a cryogenic device, typically achieved by cooling a portion of the cryogenic device to a very low temperature. Cooling can be achieved through injection of high-pressure refrigerant into an expansion chamber, such as a balloon, of the cryogenic device. Supplying refrigerant to an expandable balloon coupled to the cryogenic device may serve to expand the balloon near the target tissue for the purpose of positioning the balloon and cool the target tissue proximal to the balloon to cold-treat adjacent tissue.

### SUMMARY

If the balloon serving as an expansion chamber of a cryogenic device develops a crack, leak, rupture, or other critical structural integrity failure, coolant may escape from the cryogenic device and into the bloodstream. To address this issue, some cryogenic devices may employ a plurality of balloons where an inner balloon serving as the expansion chamber is disposed in an outer balloon such that even if the inner balloon ruptures, the coolant is still contained within the outer balloon. While such cryogenic devices clearly improve upon patient safety, breach of the inner and outer balloons may occur. Such a double balloon breach may be undesirable due to potential leakage of the coolant into the patient's bloodstream.

In accordance with one or more aspects of this disclosure, a cryogenic system may detect and/or mitigate a double balloon breach. For instance, the cryogenic system may measure parameters of the cryogen, such as the pressure, temperature, flow rate, etc., flowing through a catheter of the cryogenic system. Based on the parameters, the cryogenic system may determine whether one or more breach conditions are satisfied, where satisfaction of any of the breach conditions indicates that the plurality of balloons are breached.

Responsive to determining that one or more breach conditions are satisfied, the cryogenic system may cause, at a first time, suction to be exerted on one or more vacuum lines of the cryogenic system. The suction may be due to passive and/or active scavenging. For example, a passive suction source may passively exert suction on a first vacuum line to reverse the flow of cryogen and prevent (or at least reduce the amount of) cryogen from going into and leaking through the breached balloons. Simultaneously, an active suction source (e.g., a vacuum pump) may actively exert suction on a second vacuum line to remove cryogen from within the inner balloon and the portion of the hydraulic circuit between the breached balloons and the active suction source. Exerting passive suction on the first vacuum line and active suction on the second vacuum line in this manner may constitute a partial vacuum.

Additionally, responsive to determining that at least one breach condition is satisfied, the cryogenic system may cause, at a second time that is after the first time (e.g., within 600 milliseconds after the first time), the active suction source to exert active suction on both the first vacuum line and the second vacuum line. For example, responsive to determining that at least one breach condition is satisfied, the cryogenic system may activate a shunt (e.g., a solenoid valve) between the first and second vacuum lines such that the second vacuum source is simultaneously exerting active suction on the first vacuum line and the second vacuum line. Exerting active suction on both the first vacuum line and second vacuum line (e.g., through the shunt) may constitute a full vacuum. Applying a full vacuum at the second time (e.g., when most of the cryogen has been removed from the catheter of the cryogenic system) may advantageously allow for the remaining fluid cryogen to be quickly removed from the catheter (e.g., when avoiding worsening the breach is no longer a high priority). In this way, the cryogenic system may detect breach of the balloons and/or prevent coolant from escaping into the bloodstream.

In one example, a system comprises a catheter comprising a plurality of balloons that at least comprises an outer balloon and an inner balloon positioned within the outer balloon; a plurality of sensors, each sensor of the plurality of sensors configured to measure a corresponding parameter of a plurality of parameters; and a console comprising: a first vacuum line and a second vacuum line, each fluidly coupling the console and the catheter; and processing circuitry configured to: determine whether at least one breach condition of a plurality of breach conditions is satisfied based on at least one of the plurality of parameters, wherein satisfaction of at least one of the breach conditions indicates breach of the plurality of balloons; and responsive to determining that at least one of the breach conditions is satisfied: cause, at a first time, passive suction to be exerted on the first vacuum line and active suction to be exerted on the second vacuum line; and cause, at a second time that is after the first time, active suction to be exerted on the first vacuum line and the second vacuum line.

In one example, a method comprises: delivering cryogenic therapy to a patient via a system comprising: a catheter comprising a plurality of balloons that at least comprises an outer balloon and an inner balloon positioned within the outer balloon; a plurality of sensors, each sensor of the plurality of sensors configured to measure a corresponding parameter of a plurality of parameters; and a console comprising: a first vacuum line and a second vacuum line, each fluidly coupling the console and the catheter; and processing circuitry; determining, by the processing circuitry, whether at least one breach condition of a plurality of breach conditions is satisfied based on at least one of the plurality of parameters, wherein satisfaction of at least one of the breach conditions indicates breach of the plurality of balloons; and responsive to the processing circuitry determining that at least one of the breach conditions is satisfied: causing, by the processing circuitry and at a first time, suction to be exerted on the first vacuum line; and causing, by the processing circuitry and at a second time that is after the first time, suction to be exerted on the first vacuum line and the second vacuum line.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example system for detecting breach of a plurality of balloons, in accordance with one or more aspects of this disclosure.

FIG. 2 is a conceptual diagram illustrating an example catheter comprising a plurality of balloons, in accordance with one or more aspects of this disclosure.

FIG. 3 is a block diagram illustrating an example console, in accordance with one or more aspects of this disclosure.

FIG. 4 is a block diagram illustrating an example console, in accordance with one or more aspects of this disclosure.

FIGS. 5A-5C are line graphs illustrating example parameters satisfying the corresponding breach conditions, in accordance with one or more techniques of this disclosure.

FIG. 6 is a flowchart illustrating an example technique for detecting breach of a plurality of balloons, in accordance with one or more techniques of this disclosure.

### DETAILED DESCRIPTION

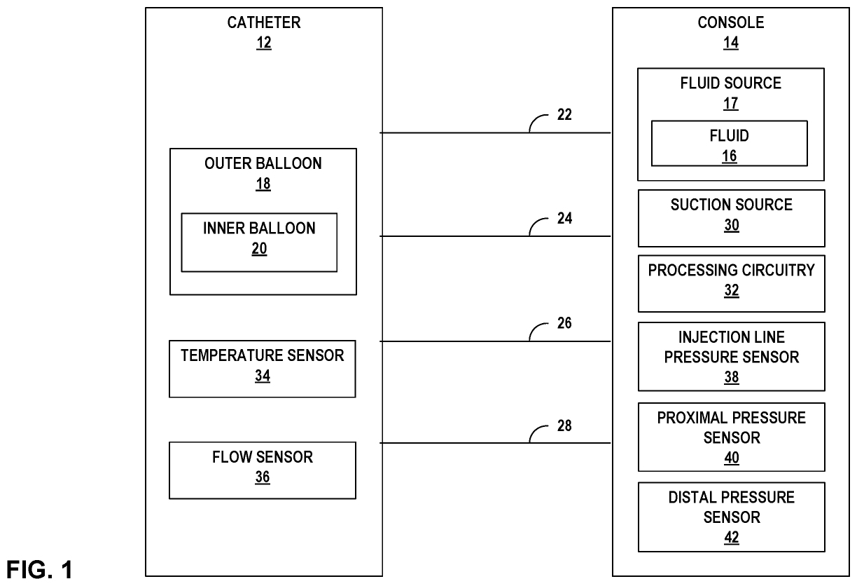

FIG. 1 is a conceptual diagram illustrating an example system 10 for detecting balloon breach that includes a catheter 12 and a console 14. System 10 may be configured to deliver cryogenic therapy. In general, to cold-treat a patient (e.g., via delivery of cryogenic therapy), a practitioner (e.g., cardiologist, surgeon, etc.) may insert catheter 12 of system 10 into a patient and use console 14 to control the flow of a fluid 16, such as cryogens, through catheter 12. In some examples, the configuration of catheter 12 and console 14 may regulate the magnitude and quality of the flow of fluid 16 through catheter 12. As fluid 16 flows through catheter 12, fluid 16 may undergo thermodynamic changes (e.g., expansion), resulting in a net transfer of heat from the target tissue to catheter 12.

As shown in FIG. 1, catheter 12 may include a plurality of balloons. The plurality of balloons may include an outer balloon 18 and an inner balloon 20 positioned within outer balloon 18. A distal portion of catheter 12 may mechanically support outer balloon 18 and inner balloon 20. In some examples, a vacuum may be maintained in the space between the inner surface of outer balloon 18 and the outer surface of inner balloon 20 for safety reasons. For instance, the vacuum between the inner surface of outer balloon 18 and the outer surface of inner balloon 20 may capture fluid 16 escaping through any breach of inner balloon 20.

Catheter 12 and console 14 may be coupled via one or more lines. For instance, as shown in FIG. 1, catheter 12 may be coupled to console 14 via an injection line 22, first vacuum line 24, a second vacuum line 26, an electrical line 28, etc. Injection line 22 may fluidly couple catheter 12 and console 14 (e.g., establish fluid communication between catheter 12 and console 14 via injection line 22) and be configured to enable injection (e.g., ingress) of fluid 16 from console 14 and catheter 12. First vacuum line 24 may fluidly couple catheter 12 and console 14 (e.g., establish fluid communication between catheter 12 and console 14 via first vacuum line 24) and be configured to enable removal (e.g., egress) of fluid 16 from catheter 12 to console 14 (e.g., as a result of suction being exerted on first vacuum line 24). Second vacuum line 26 may fluidly couple catheter 12 and console 14 (e.g., establish fluid communication between catheter 12 and console 14 via second vacuum line 26) and be configured to enable removal of fluid 16 from catheter 12 to console 14 (e.g., as a result of suction being exerted on second vacuum line 26). Electrical line 28 may electrically couple catheter 12 and console 14.

It should be understood that FIG. 1 is merely an example and should not be construed as limiting. That is, console 14 may include more or fewer lines. Moreover, although not shown as such in FIG. 1, two or more of the lines may be coaxial. For instance, injection line 22 and first vacuum line 24 may share the same line.

Console 14 may supply fluid 16 to catheter 12. In some examples, console 14 may provide fluid 16 from a fluid source 17 within console 14. For instance, fluid 16 may flow through tubing of console 14 and into catheter 12 via injection line 22. Fluid 16 may flow into inner balloon 20 and undergo endothermic reactions (e.g., expansion and changing phases from a fluid to a gas) that result in a distal tip of catheter 12, inner balloon 20, and/or outer balloon 18 freezing.

System 10 may exert suction to remove fluid 16 from outer balloon 18 and/or inner balloon 20. For example, suction sources 30 may exert suction on first vacuum line 24 and second vacuum line 26. Suction sources 30 may perform passive scavenging and/or active scavenging. For instance, an active suction source (e.g., a pump within console 14, an external pump, etc.) may exert suction on second vacuum line 26 to actively remove fluid 16 (thereby defining what may be referred to as “active suction”) from inner balloon 20 during operation of system 10. In such examples, the hydraulic circuit (or, in other words, fluidic circuit) of system 10, which includes catheter 12 and console 14, may be configured such that the active suction source removes fluid 16 that has already flowed through inner balloon 20 via second vacuum line 26. In other words, second vacuum line 26 may be distal to (or after) inner balloon 20 (as well as outer balloon 18) in the hydraulic/fluidic circuit.

Other vacuum lines may provide an emergency evacuation path for fluid 16 from catheter 12 if necessary. For instance, in the event that inner balloon 20 is breached, one or more of suction sources 30 may exert suction on first vacuum line 24 and second vacuum line 26. In some examples, the hydraulic circuit of system 10 may be configured such that suction sources 30 remove fluid 16 that has not yet flowed through inner balloon 20 via first vacuum line 24 and removes fluid that has already flowed through inner balloon 20 via second vacuum line 26.

System 10 may include processing circuitry 32 configured to perform techniques in accordance with this disclosure. Processing circuitry 32 may include fixed function circuitry and/or programmable processing circuitry. Processing circuitry 32 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 32 may include multiple components, such as any combination of one or more microprocessors, one or more controllers, one or more DSPs, one or more ASICs, or one or more FPGAs, as well as other discrete or integrated logic circuitry. The functions attributed to processing circuitry 32 herein may be embodied as software, firmware, hardware or any combination thereof.

System 10 may include a plurality of sensors, each sensor configured to measure a corresponding parameter of the plurality of parameters. The plurality of sensors may include one or more of any suitable temperature sensor, any suitable pressure sensor, or any suitable flowmeter. Examples of temperature sensors may include a thermocouple, a thermistor, a junction-based thermal sensor, a thermopile, a fiber optic detector, an acoustic temperature sensor, a quartz or other resonant temperature sensor, a thermo-mechanical temperature sensor, a thin film resistive element, etc. Examples of pressure sensors may include a differential pressure sensor, a pressure transducer, a piezometer, etc. Examples of flowmeters may include a differential pressure flow meter, a positive displacement flow meter, a velocity flow meter, a mass flow meter, an open channel flow meter, etc. Processing circuitry 32 may receive or otherwise obtain the parameters from the sensors as inputs.

In general, the sensors may be configured to monitor parameters of fluid 16 as fluid travels through the hydraulic circuit. The sensors may be located in various locations throughout system 10. For example, as shown in FIG. 1, catheter 12 may include a temperature sensor 34 and a flow sensor 36, and console 14 may include an injection line pressure sensor 38, a proximal pressure sensor 40, and a distal pressure sensor 42.

Temperature sensor 34 may be configured to measure a temperature parameter. For example, temperature sensor 34 may be configured to measure a temperature parameter by measuring a temperature within inner balloon 20. In some examples, temperature sensor 34 may be positioned within catheter 12 (e.g., disposed within inner balloon 20).

Flow sensor 36 may be configured to measure a flow parameter. For example, flow sensor 36 may be configured to measure a flow parameter by measuring a flow rate of expanded fluid 16 within the second vacuum line during passive scavenging.

Injection line pressure sensor 38 may be configured to measure an injection line pressure parameter. For example, injection line pressure sensor 38 may be configured to measure an injection line pressure parameter by measuring a pressure within injection line 22. In some examples, injection line pressure sensor 38 may be positioned within console 14 (e.g., disposed within a portion of injection line 22 disposed within console 14).

Proximal pressure sensor 40 may be configured to measure a proximal pressure parameter. For example, proximal pressure sensor 40 may be configured to measure a proximal pressure parameter by measuring a pressure within a proximal portion of second vacuum line 26. In some examples, proximal pressure sensor 40 may be positioned within console 14 (e.g., disposed within a portion of second vacuum line 26 disposed within console 14). In some examples, proximal pressure sensor 40 may monitor pressure within outer balloon 18 and inner balloon 20 during the inflation phase to prevent over pressurization. An inflation source (which may include, e.g., a vacuum proportional valve) may use the pressure within outer balloon 18 and inner balloon 20 as feedback during the transition phase to maintain the inflated configuration of outer balloon 18 and inner balloon 20. In some examples, system 10 (e.g., processing circuitry 32) may monitor the mechanical connection between catheter 12 and console 14 based on signals from flow sensor 36 and proximal pressure sensor 40.

Distal pressure sensor 42 may be configured to measure a distal pressure parameter. For example, distal pressure sensor 42 may be configured to measure a distal pressure parameter by measuring a pressure within a distal portion of second vacuum line 26. Proximal pressure sensor 40 may be more proximal than distal pressure sensor 42 in that fluid removed from catheter 12 via second vacuum line 26 reaches proximal pressure sensor 40 before distal pressure sensor 42. In some examples, distal pressure sensor 42 may be positioned within console 14 (e.g., disposed within a portion of second vacuum line 26 disposed within console 14). In some examples, distal pressure sensor 42 may monitor second vacuum line 26 to detect failure of suction source 30 or obstruction of second vacuum line 26.

If both outer balloon 18 and inner balloon 20 develops a crack, leak, rupture, or other critical structural integrity failure (generally referred to herein as a “double balloon breach” or “DBB”), fluid 16 may escape from catheter 12 and into the bloodstream of a patient. DBB can occur at different phases during an operation of system 10. For example, DBB may occur during a transition phase (e.g., a transition from inflation to ablation) when console 14 gradually increases the pressure of fluid.

In accordance with one or more aspects of this disclosure, system 10 may be configured to detect breach of outer balloon 18 and inner balloon 20 and prevent fluid 16 from escaping into the bloodstream. In particular, system 10 may include processing circuitry 32 configured to determine whether at least one breach condition of a plurality of breach conditions is satisfied, where satisfaction of at least one of the breach conditions indicates DBB. Responsive to determining that at least one of the breach conditions is satisfied, processing circuitry 32 may be configured to cause, at a first time, a passive suction source to exert suction on first vacuum line 24 to passively remove fluid 16 (thereby defining what may be referred to as “passive suction”) from first vacuum line 24. Also at the first time, an active suction source may exert active suction on second vacuum line 26. Exerting passive suction on first vacuum line 24 and active suction on second vacuum line 26 may constitute a partial vacuum. Additionally, responsive to processing circuitry 32 determining that at least one breach condition is satisfied, processing circuitry 32 may be configured to cause, at a second time that is after the first time (e.g., within 600 ms of the first time), active suction to be exerted on both first vacuum line 24 and second vacuum line 26. Applying a partial vacuum at a first time may advantageously reduce the likelihood of further damage to outer balloon 18 and inner balloon 20 due to scavenging of fluid 16. Applying a full vacuum at the second time (e.g., when most of fluid 16 has been removed from catheter 12) may advantageously allow for the remaining fluid 16 to be removed from catheter 12. Accordingly, the techniques of this disclosure may enable rapid detection and prevention of escaping fluid 16, thereby improving patient safety.

As noted above, processing circuitry 32 may be configured to determine satisfaction of at least one breach condition based on at least one parameter. For example, processing circuitry 32 may be configured to determine whether a first temperature breach condition is satisfied by the temperature parameter measured by temperature sensor 34. In some examples, processing circuitry 32 may be configured to determine that the first temperature breach condition is satisfied when the temperature parameter is less than or equal to a temperature threshold value. For instance, if the temperature parameter is −18 degrees Celsius (° C.) and the temperature threshold value is −30° C., then processing circuitry 32 may determine that the first temperature breach condition is not satisfied. However, if in the above example the temperature parameter is instead −37° C., then processing circuitry 32 may determine that the first temperature breach condition is satisfied.

Temperature threshold values other than −30° C. are contemplated. For instance, the temperature threshold value may be selected to make satisfaction of the first temperature breach condition occur more quickly in response to DBB (e.g., by making the temperature threshold value closer to the expected value of the temperature parameter during operation of system 10). In some examples, the temperature threshold value may change in a predetermined manner as a function of time (e.g., a predetermined temperature threshold value 100 seconds after implantation of catheter 12 may be different from a predetermined temperature threshold value 200 seconds after implantation) and/or phase (e.g., inflation phase, transition phase, ablation phase, etc.) of operation of system 10. The first temperature breach condition described in the above example may be particularly useful during the ablation phase when the temperature of fluid 16 is expected to be relatively constant.

In another example, processing circuitry 32 may be configured to determine whether a second temperature breach condition is satisfied by the temperature parameter measured by temperature sensor 34. In some examples, processing circuitry 32 may be configured to determine that the second temperature breach condition is satisfied when a slope of the temperature parameter is less than or equal to a temperature slope threshold value. For instance, if the slope of the temperature parameter is about −1° C. per second (° C./s) and the temperature slope threshold value is −5° C./s, then processing circuitry 32 may determine that the second temperature breach condition is not satisfied. However, if in the above example the temperature parameter is instead −15° C./s, then processing circuitry 32 may determine that the second temperature breach condition is satisfied.

Temperature slope threshold values other than −5° C./s are contemplated. For instance, the temperature slope threshold value may be selected to make satisfaction of the second temperature breach condition occur more quickly in response to DBB (e.g., by making the temperature slope threshold value closer to the expected slope of the temperature parameter during operation of system 10). In some examples, the temperature slope threshold value may change in a predetermined manner as a function of time and/or phase of operation of system 10. The second temperature breach condition described in the above example may be particularly useful during the ablation phase when the temperature of fluid 16 is expected to be stable (i.e., have a slope of about 0).

It should be understood that the temperature of fluid 16 may trend upward, downward, or sideways based on a phase of operation of system 10. For example, during the transition phase, the temperature of fluid 16 may consistently trend downward. In that case, the temperature parameter and/or a slope of the temperature parameter may satisfy the respective temperature breach conditions when the temperature parameter and/or the slope of the temperature parameter are greater than or equal to the respective threshold values. Thus, in general (e.g., with respect to the temperature parameter as well as to other parameters described herein), processing circuitry 32 may determine that a breach condition is satisfied when one or more parameter values change (e.g., by an amount exceeding a predetermined threshold amount) in a direction contrary to the respective expected trends of those parameters based on the phase of operation of system 10.

In another example, processing circuitry 32 may be configured to determine whether a first flow breach condition is satisfied by the flow parameter measured by flow sensor 36. In some examples, processing circuitry 32 may be configured to determine that the first flow breach condition is satisfied when the flow parameter is less than or equal to a flow threshold value. For instance, if the flow parameter is 6200 standard cubic centimeters per minute (sccm) and the flow threshold value is 6150 sccm, then processing circuitry 32 may determine that the first flow breach condition is not satisfied. However, if in the above example the flow parameter is instead 6149 sccm, then processing circuitry 32 may determine that the first flow breach condition is satisfied.

Flow threshold values other than 6200 sccm are contemplated. For instance, the flow threshold value may be selected to make satisfaction of the first flow breach condition occur more quickly in response to DBB (e.g., by making the flow threshold value closer to the expected value of the flow parameter during operation of system 10). In some examples, the flow threshold value may change in a predetermined manner as a function of time and/or phase of operation of system 10. The first flow breach condition described in the above example may be particularly useful during the ablation phase when the flow rate of fluid 16 is expected to be relatively constant.

In another example, processing circuitry 32 may be configured to determine whether a second flow breach condition is satisfied by the flow parameter measured by flow sensor 36. In some examples, processing circuitry 32 may be configured to determine that the second flow breach condition is satisfied when a slope of the flow parameter is less than or equal to a flow slope threshold value. For instance, if the slope of the flow parameter is about 200 sccm/s and the flow slope threshold value is −5 sccm/s, then processing circuitry 32 may determine that the second flow breach condition is not satisfied. However, if in the above example the flow parameter is instead −100 sccm/s, then processing circuitry 32 may determine that the second flow breach condition is satisfied.

Flow slope threshold values other than −5 sccm/s are contemplated. For instance, the flow slope threshold value may be selected to make satisfaction of the second flow breach condition occur more quickly in response to DBB (e.g., by making the flow slope threshold value closer to the expected slope of the flow parameter during operation of system 10). In some examples, the flow slope threshold value may change in a predetermined manner as a function of time and/or phase of operation of system 10. The second temperature breach condition described in the above example may be particularly useful during the transition phase when the temperature of fluid 16 is expected to consistently trend upward (i.e., have a positive slope) until reaching a high flow rate value at the end of the transition phase.

In another example, processing circuitry 32 may be configured to determine whether an injection line breach condition is satisfied based on the injection line pressure parameter measured by injection line pressure sensor 38. In some examples, processing circuitry 32 may be configured to determine that the injection line breach condition is satisfied when a slope of the injection line pressure parameter is greater than or equal to an injection line pressure slope threshold value. For instance, if the slope of the injection line pressure parameter is about 10 psig/s and the injection pressure slope threshold value is 80 psig/s, then processing circuitry 32 may determine that the injection line pressure breach condition is not satisfied. However, if in the above example the injection line pressure parameter is instead 100 psig/s, then processing circuitry 32 may determine that the injection line pressure breach condition is satisfied.

Injection line pressure slope threshold values other than 80 psig/s are contemplated. For instance, the injection line pressure threshold value may be selected to make satisfaction of the injection line pressure breach condition occur more quickly in response to DBB (e.g., by making the injection pressure slope threshold value closer to the expected injection line pressure slope of fluid 16 during operation of system 10). In some examples, the injection line pressure slope threshold value may change in a predetermined manner as a function of time and/or phase of operation of system 10.

In another example, processing circuitry 32 may be configured to determine whether a concurrent pressure change breach condition is satisfied based on the proximal pressure parameter measured by proximal pressure sensor 40 and the distal pressure parameter measured by distal pressure sensor 42. In some examples, processing circuitry 32 may be configured to determine that the concurrent pressure change breach condition is satisfied when, within a predetermined period of time (e.g., about 10 milliseconds (ms)): a slope of the proximal pressure parameter decreases by an amount greater than or equal to a proximal pressure change threshold value; and a slope of the distal pressure parameter decreases by an amount greater than or equal to a distal pressure change threshold value.

As an example, at t₁=0 ms, the slope of the proximal pressure parameter may decrease by 6 psi absolute per second (psia/s), and, at t₂=20 ms, the slope of the distal pressure parameter may decrease by 10 psia/s. If the proximal pressure change threshold value is 5 psia/s, the distal pressure change threshold value is 5 psia/s, and the predetermined period of time is 10 ms, then processing circuitry 32 may determine that the concurrent pressure change breach condition is not satisfied (because the changes in slopes of the proximal pressure parameter and the proximal pressure parameter did not occur within the predetermined period of time of 10 ms). However, if in the above example t₂ is instead 5 ms and the predetermined period of time is still 10 ms, then processing circuitry 32 may determine that the concurrent pressure change breach condition is satisfied.

Proximal pressure change threshold values other than 5 psia/s, distal pressure change threshold values other than 5 psia/s, and predetermined periods of time other than 10 ms are contemplated. For instance, the proximal pressure change threshold value, distal pressure change threshold value, and/or predetermined period of time may be selected to make satisfaction of the concurrent pressure change breach condition occur more quickly in response to DBB. In some examples, the proximal pressure change threshold value, distal pressure change threshold value, and/or predetermined period of time may change in a predetermined manner as a function of time and/or phase of operation of system 10.

In another example, processing circuitry 32 may be configured to execute a machine learning algorithm configured to determine whether at least one of the plurality of breach conditions (including, but not limited to, the breach conditions described above) is satisfied based on at least one of the plurality of parameters. In other words, the machine learning algorithm may be configured to detect an occurrence of DBB. The machine learning algorithm may receive the plurality of parameters measured by the sensors of system 10 as input data. In some examples, the machine learning algorithm may perform various types of classification based on the input data. For example, the machine learning algorithm may perform binary classification. In binary classification, the output data may include a classification of the input data into one of two different classes, such as “DBB” or “no DBB.” In other examples, the machine learning algorithm may perform multiclass classification. In multiclass classification, the output data may include a classification of the input data into one of three or more different classes, such as “DBB,” “low risk of DBB,” “moderate risk of DBB,” or “high risk of DBB.”

In some examples, the machine learning algorithm may perform classification in which the machine learning algorithm provides, for each of one or more classes, a numerical value descriptive of a degree to which it is believed that the input data should be classified into the corresponding class. In some instances, the numerical values provided by the machine learning algorithm can be referred to as “confidence scores” that are indicative of a respective confidence associated with classification of the input into the respective class. In some examples, only a certain number of classes (e.g., one) with the relatively largest confidence scores can be selected to render a discrete categorical prediction.

In any case, responsive to processing circuitry 32 determining that at least one breach condition is satisfied, processing circuitry 32 may be configured to cause, at a first time, passive suction to be exerted on first vacuum line 24 (e.g., by a passive suction source of suction sources 30) and active suction to be exerted on second vacuum line 26 (e.g., by an active suction source of suction sources 30). Additionally, responsive to processing circuitry 32 determining that at least one breach condition is satisfied, processing circuitry 32 may be configured to cause, at a second time that is after the first time (e.g., within 600 ms of the first time), active suction to be exerted on both first vacuum line 24 and second vacuum line 26. For example, at or shortly prior to the second time, processing circuitry 32 may activate a shunt valve (e.g., a solenoid valve) such that the active suction source exerts active suction on both first vacuum line 24 and second vacuum line 26.

Exerting active suction on first vacuum line 24 and second vacuum line 26 may constitute applying a full vacuum to catheter 12. The suction force of a partial vacuum may be less than the suction force of a full vacuum, which may advantageously reduce the likelihood of further damage to outer balloon 18 and inner balloon 20 due to scavenging of fluid 16. However, applying a partial vacuum may be less likely to cause complete drain of outer balloon 18, inner balloon 20, and injection line 22 than applying a full vacuum. Thus, applying a full vacuum at the second time (e.g., when most of fluid 16 has been removed from catheter 12) may advantageously allow for the remaining fluid 16 to be removed from catheter 12. In general, both the first time and the second time may occur within a few seconds (e.g., about 2 to 3 seconds) of DBB, and preferably within less than a second (e.g., within 600 ms). Accordingly, the techniques of this disclosure may enable rapid detection and prevention of escaping fluid 16, thereby improving patient safety.

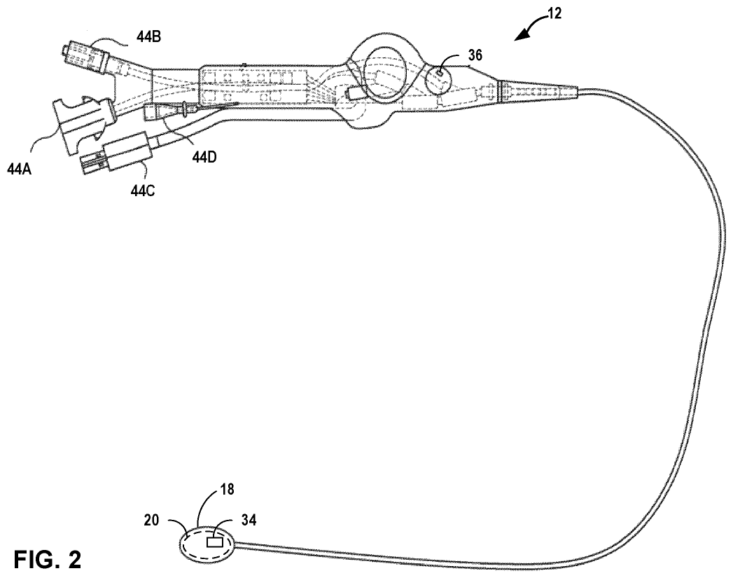

FIG. 2 is a conceptual diagram illustrating catheter 12. Catheter 12 includes a handle having proximal connector ports 44A-44D. Connector port 44A may be a coaxial connector having both a first vacuum lumen (e.g., an 8 French lumen) and an injection lumen therein. Injection line 22 and first vacuum line 24 may connect to connector port 44A. Connector port 44B may be a second vacuum connector, having a second vacuum lumen (e.g., 10 French lumen) therein. Second vacuum line 26 may connect to connector port 44B. Connector port 44C may be an electrical connector. Electrical line 28 may connect to connector port 44C. Connector port 44D may be a guidewire luer hub.

A distal portion of catheter 12 may include a plurality of balloons, such as outer balloon 18 and inner balloon 20 positioned within outer balloon 18. A soft distal tip may be located distal to outer balloon 18 and inner balloon 20. When fluid 16 is injected into outer balloon 18 and inner balloon 20, suction applied through connector ports 44A-44B may draw any fluid within outer balloon 18 and inner balloon 20 out of outer balloon 18 and inner balloon 20 and catheter 12. Temperature sensor 34 may be disposed within inner balloon 20. In some examples, radiopaque marker bands may be located proximate to the exit point of fluid 16 injected into inner balloon 20 to aid in the positioning and tracking of catheter 12.

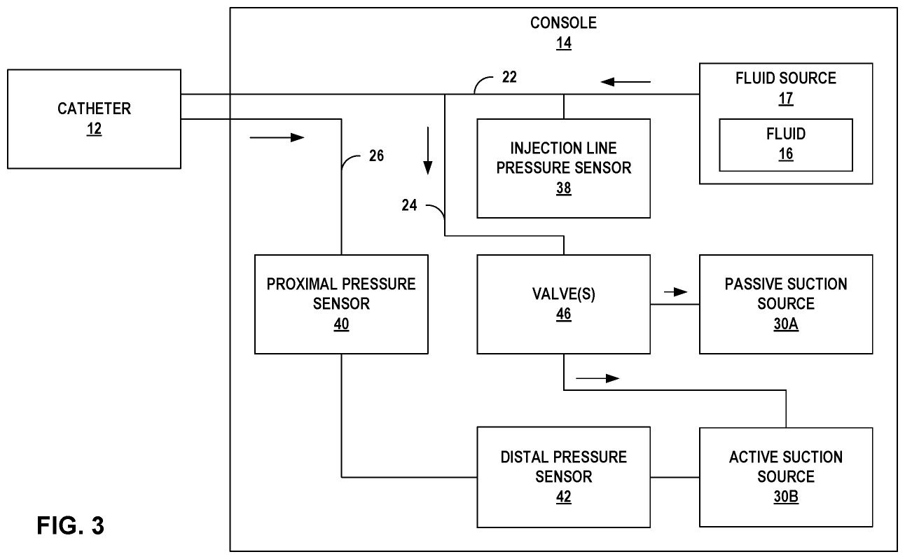

FIG. 3 is a block diagram illustrating console 14. As shown in the example of FIG. 3, console 14 includes fluid source 17, a passive suction source 30A (e.g., an exhaust of console 14), an active suction source 30B, injection line pressure sensor 38, proximal pressure sensor 40, distal pressure sensor 42, and one or more valves 46. Console 14 may provide fluid 16 to catheter 12 via injection line 22 and receive fluid 16 from catheter 12 via second vacuum line 26.

Valves 46, such as solenoid valves, may be disposed within various lines of console 14. Depending on the phase of operation of console 14, console 14 may open (e.g., activate, actuate, etc.) or close (e.g., deactivate) valves 46. For example, during the inflation phase, a first set of valves may be open and a second set of valves may be closed, and during the transition phase, the first set of valves may be closed and the second set of valves may be open. By opening and closing select valves, console 14 may regulate the pressure, flow, etc., of fluid 16, particularly within inner balloon 20. Examples of valves 46 may include solenoid valves. In some examples, valves 46 may include a vacuum proportional valve configured to facilitate flow of fluid 16 through injection line 22.

Injection line pressure sensor 38 may be disposed within injection line 22 to measure the injection line pressure parameter by measuring the pressure within injection line 22. Proximal pressure sensor 40 may be disposed within second vacuum line 26 to measure the proximal pressure parameter by measuring the pressure within a proximal portion of second vacuum line 26. Distal pressure sensor 42 may be disposed within second vacuum line 26 to measure the distal pressure parameter by measuring the pressure within a distal portion of second vacuum line 26.

Processing circuitry 32 may be configured to control valves 46 in accordance with one or more techniques of this disclosure. For instance, at the first time (i.e., when passive suction source 30A is supposed to be exerting passive suction on first vacuum line 24 and active suction source 30B is supposed to be exerting active suction on second vacuum line 26), one or more valves 46 may be closed such that passive suction source 30A, but not active suction source 30B, is coupled to first vacuum line 24. That is, when system 10 is in this state, valves 46 may allow passive suction source 30A to exert passive suction on first vacuum line 24 while preventing active suction source 30B from exerting active suction on first vacuum line 24. At the second time (i.e., when active suction source 30B is supposed to be exerting active suction on first vacuum line 24 and second vacuum line 26), one or more valves 46 may open (e.g., activated, actuated, etc.) such that active suction source 30B, but not passive suction source 30A, is coupled to first vacuum line 24 and second vacuum line 26. That is, when system 10 is in this state, valves 46 may allow active suction source 30B to exert active suction on first vacuum line 24 while preventing passive suction source 30A from exerting active suction on first vacuum line 24.

In some examples, responsive to determining that at least one of the breach conditions is satisfied, processing circuitry 32 may cause, at the first time, injection of fluid 16 to be stopped and cause, at the first time, suction exerted on second vacuum line 26 to be stopped. In such examples, one or more valves may be disposed within second vacuum line 26, and processing circuitry 32 may open and close those valves to enable or disable suction to be exerted on second vacuum line 26, respectively. In some examples, responsive to determining that at least one of the breach conditions is satisfied, processing circuitry 32 may generate one or more alerts. For example, responsive to determining that at least one of the breach conditions is satisfied, processing circuitry 32 may generate an alert notifying a user (e.g., a clinician) to monitor gas egress using fluoroscopy. Processing circuitry 32 may also generate an alert notifying a user to be careful when retracting catheter 12 to prevent fragments of outer balloon 18 and/or inner balloon 20 from escaping into the vasculature of the patient.

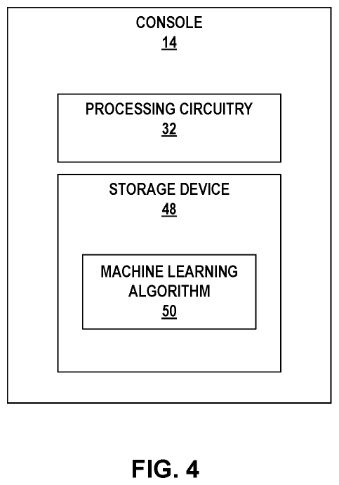

FIG. 4 is a block diagram illustrating console 14, in accordance with one or more aspects of this disclosure. As shown in FIG. 4, console 14 includes processing circuitry 32 and a storage device 48. As further shown in FIG. 4, storage device 48 includes a machine learning algorithm 50 (e.g., the machine learning algorithm described with respect to FIG. 1).

Processing circuitry 32 may include one or more processors, such as any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 32 herein may be embodied as firmware, hardware, software or any combination thereof.

Storage device 48 may be configured to store information within console 14, respectively, during operation. Storage device 48 may include a computer-readable storage medium or computer-readable storage device. In some examples, storage device 48 includes one or more of a short-term memory or a long-term memory. Storage device 48 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 48 is used to store data indicative of instructions, e.g., for execution by processing circuitry 32, respectively. Storage device 48 may be configured to store the various threshold values, parameters, etc.

Machine learning algorithm 50 may be operable (or, in other words, executed) by processing circuitry 32 to perform various actions, operations, or functions of system 10. In some examples, machine learning algorithm 50 may be trained locally and/or remotely. For example, a remote computing system may execute a training module configured to train machine learning algorithm 50 based on training data. The training data may include input data including the parameters from the sensors of system 10 and/or systems similar to system 10.

In some examples, the training module may train machine learning algorithm 50 using supervised learning techniques. For example, the training module may train machine learning algorithm 50 on training data that includes training examples of input data labeled as belonging (or not belonging) to the “DBB” class or “not DBB” class. Responsive to machine learning algorithm 50 being trained, machine learning algorithm 50 may classify input data including parameters measured by sensors of system 10 as “DBB,” “not DBB,” etc. Processing circuitry 32 may determine that a machine learning breach condition is satisfied when machine learning algorithm 50 classifies input data as DBB. In some examples, the classification of input data as DBB may be based on machine learning algorithm 50 determining that one or more other breach conditions, including but not limited to the breach conditions explicitly described herein (e.g., first and second temperature breach conditions, first and second flow breach conditions, concurrent pressure change breach condition, etc.), are satisfied.

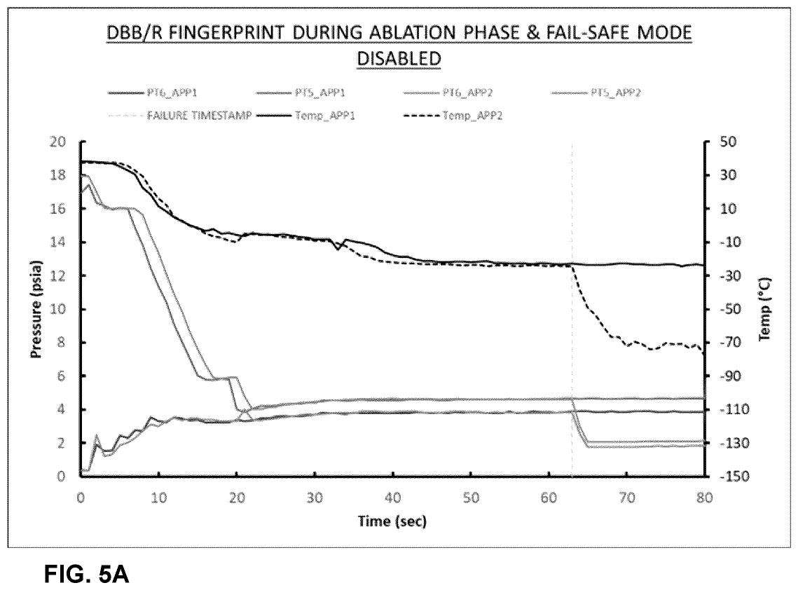

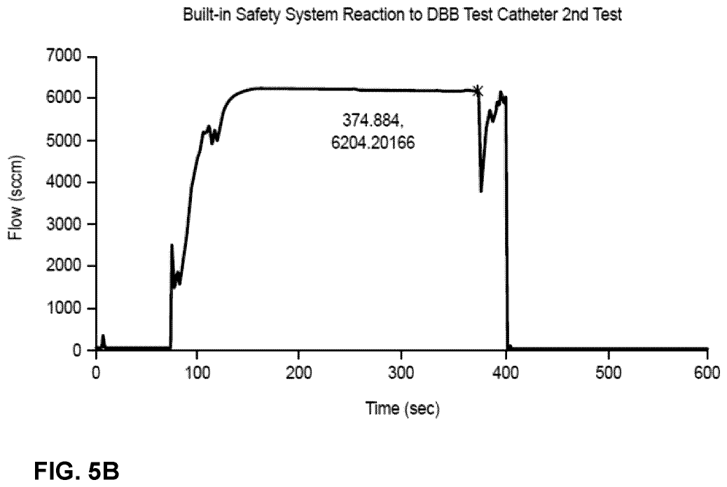

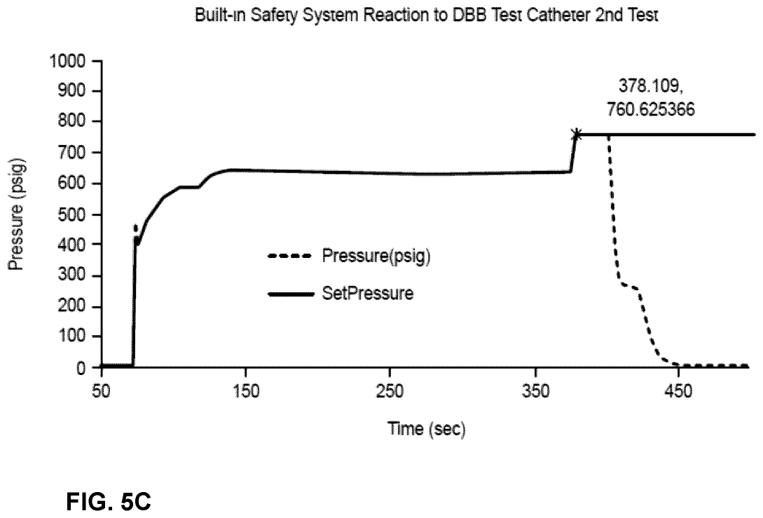

FIGS. 5A-5C are line graphs illustrating example parameters satisfying the corresponding breach conditions. FIG. 5A illustrates satisfaction of the first temperature breach condition and/or second temperature breach condition. At about 63 sec, the temperature measured by temperature sensor 34 decreased sharply from about −25° C., potentially indicating DBB. FIG. 5B illustrates satisfaction of the first flow breach condition and/or second flow breach condition. At 374.884 sec, the flow rate measured by flow sensor 36 decreased sharply from 6204.20166 standard cubic centimeters per minute (sccm), potentially indicating occurrence of DBB. FIG. 5C illustrates satisfaction of the concurrent pressure change breach condition. At 376.345 sec, the pressure measured by proximal pressure sensor 40 intersected the pressure measured by distal pressure sensor 42, potentially indicating occurrence of DBB.

Responsive to determining that at least one breach condition is satisfied, processing circuitry 32 may be configured to cause, at a first time, passive suction source 30A to exert passive suction on first vacuum line 24 and active suction source 30B to exert active suction on second vacuum line 26. For example, within milliseconds of satisfaction of the first flow breach condition and/or second flow breach condition at 374.884 sec, processing circuitry 32 may cause: (one or more) valves 46 to open and/or close (such that passive suction source 30A is in fluid communication with first vacuum line 24), injection of fluid 16 to be stopped, and passive suction of first vacuum line 24 and active suction of second vacuum line 26 to be started. At the second time, processing circuitry 32 may cause (one or more) valves 46 to open and/or close (such that active suction source 30B is in fluid communication with first vacuum line 24) and active suction of first vacuum line 24 and second vacuum line 26.

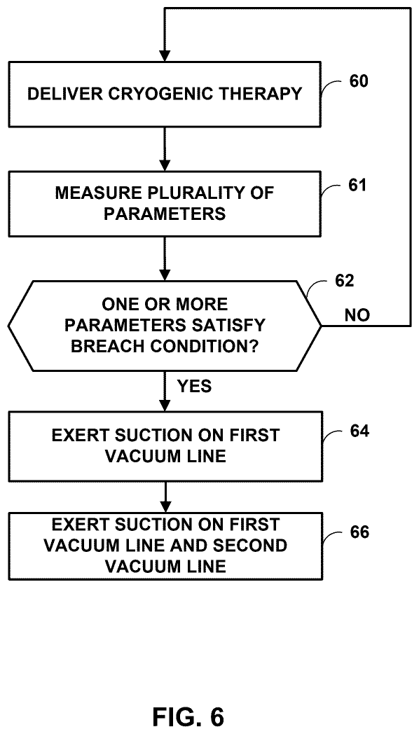

FIG. 6 is a flowchart illustrating an example technique for using system 10, in accordance with one or more techniques of this disclosure. System 10 may deliver cryogenic therapy by injecting fluid 16 into injection line 22 (60). Sensors of system 10 may measure parameters of fluid 16 as fluid travels through the hydraulic circuit (61). The sensors may be located in various locations throughout system 10. For example, catheter 12 may include temperature sensor 34 and flow sensor 36, and console 14 may include injection line pressure sensor 38, proximal pressure sensor 40, and distal pressure sensor 42 (62). For example, processing circuitry 32 may be configured to determine whether a first temperature breach condition, a second temperature breach condition, a first flow breach condition, a second flow breach condition, a pressure differential breach condition, a concurrent pressure change breach condition, a machine learning breach condition, etc., is satisfied by the parameters measured by sensors of system 10. As one example, processing circuitry 32 may be configured to determine that the first temperature breach condition is satisfied when the temperature parameter is greater than or equal to a temperature threshold value. For instance, if the temperature parameter is −38 degrees Celsius (° C.) and the temperature threshold value is −37° C., then processing circuitry 32 may determine that the first temperature breach condition is not satisfied. However, if in the above example the temperature parameter is instead −37° C., then processing circuitry 32 may determine that the first temperature breach condition is satisfied.

If processing circuitry 32 determines that none of the breach conditions are satisfied (NO of 62), sensors may continue to measure the corresponding parameters (60). If processing circuitry 32 determines that at least one of the breach conditions is satisfied (YES of 62), processing circuitry 32 may cause, at a first time, passive suction to be exerted on first vacuum line 24 and active suction to be exerted on second vacuum lien 26 (64). In some examples, in addition to causing suction to be exerted on first vacuum line 24 responsive to the at least one breach condition being satisfied, processing circuitry 32 may cause injection of fluid 16 to be stopped. After the first time (i.e., at a second time), processing circuitry 32 may cause active suction to be exerted on first vacuum line 24 and second vacuum line 26 (66).

As noted above, applying a partial vacuum instead of a full vacuum may be less likely to cause further damage to outer balloon 18 and inner balloon 20. Thus, applying a partial vacuum at the first time may advantageously allow for fluid 16 to be removed from catheter 12 while reducing a risk of increasing a size of the breach. Applying a full vacuum at the second time (e.g., when most of fluid 16 has been removed from catheter 12) may advantageously allow for the remaining fluid 16 to be removed from catheter 12 (e.g., when avoiding worsening the breach is no longer a high priority). In general, both the first time and the second time may occur within a few seconds (e.g., about 2 to 3 seconds) of DBB, if not sooner. In this way, techniques of this disclosure may enable rapid detection and prevention of escaping fluid 16, thereby improving patient safety.

The techniques described in this disclosure may be implemented, at least in part, in hardware, software, firmware or any combination thereof. For example, various aspects of the described techniques may be implemented within processing circuitry, which may include one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also form one or more processors or processing circuitry configured to perform one or more of the techniques of this disclosure.

Such hardware, software, and firmware may be implemented, and various operation may be performed within same device, within separate devices, and/or on a coordinated basis within, among or across several devices, to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components. Processing circuitry described in this disclosure, including a processor or multiple processors, may be implemented, in various examples, as fixed-function circuits, programmable circuits, or a combination thereof. Fixed-function circuits refer to circuits that provide particular functionality with preset operations. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive stimulation parameters or output stimulation parameters), but the types of operations that the fixed-function circuits perform are generally immutable. In some examples, one or more of the units may be distinct circuit blocks (fixed-function or programmable), and in some examples, one or more of the units may be integrated circuits.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

1. A system comprising:
a catheter comprising a plurality of balloons that at least comprises an outer balloon and an inner balloon positioned within the outer balloon;
a plurality of sensors, each sensor of the plurality of sensors configured to measure a corresponding parameter of a plurality of parameters; and
a console comprising:
a first vacuum line and a second vacuum line, each fluidly coupling the console and the catheter; and
processing circuitry configured to:
determine whether at least one breach condition of a plurality of breach conditions is satisfied based on at least one of the plurality of parameters, wherein satisfaction of at least one of the breach conditions indicates breach of each balloon of the plurality of balloons; and
responsive to determining that at least one of the breach conditions is satisfied:
cause, at a first time, passive suction to be exerted on the first vacuum line and active suction to be exerted on the second vacuum line; and
cause, at a second time that is after the first time, active suction to be exerted on the first vacuum line and the second vacuum line.

2. The system of claim 1, wherein the console further comprises an injection line, fluidly coupling the console and the catheter, configured to enable injection of a fluid from the console to the catheter via the injection line;
wherein optionally the plurality of parameters comprises an injection line pressure parameter, and wherein the plurality of sensors comprises an injection line pressure sensor, positioned within the console, configured to measure the injection line pressure parameter by measuring a pressure within the injection line.

3. The system of claim 2, wherein the processing circuitry is configured to determine that a concurrent pressure change breach condition of the plurality of breach conditions is satisfied when, within a predetermined period of time:
a slope of the distal pressure parameter decreases by an amount greater than or equal to a proximal pressure change threshold value; and
a slope of the proximal pressure parameter decreases by an amount greater than or equal to a proximal pressure change threshold value.

4. The system of claim 1, wherein the plurality of parameters comprises a temperature parameter, and wherein the plurality of sensors comprises a temperature sensor, positioned within the inner balloon, configured to measure the temperature parameter by measuring a temperature within the inner balloon;
wherein optionally the processing circuitry is configured to determine that a first temperature breach condition of the plurality of breach conditions is satisfied when the temperature parameter is greater than or equal to a temperature threshold value.

5. The system of claim 4, wherein the processing circuitry is configured to determine that a second temperature breach condition of the plurality of breach conditions is satisfied when a slope of the temperature parameter is greater than or equal to a temperature slope threshold value.

6. The system of claim 2, wherein the plurality of parameters comprises a flow parameter, and wherein the plurality of sensors comprises a flow sensor, positioned within the console, configured to measure the flow parameter by measuring a flow rate within the injection line;
wherein optionally the processing circuitry is configured to determine that a first flow breach condition of the plurality of breach conditions is satisfied when the flow parameter is less than or equal to a flow threshold value;
wherein optionally the processing circuitry is configured to determine that a second flow breach condition of the plurality of breach conditions is satisfied when a slope of the flow parameter is less than or equal to a flow slope threshold value.

7. The system of claim 1, wherein the processing circuitry is configured to determine whether at least one of the plurality of breach conditions is satisfied based on at least one of the plurality of parameters by executing a machine learning algorithm configured to receive the plurality of parameters as input data.

8. The system of claim 1, wherein the fluid is cryogen, and wherein the system is configured to deliver cryogenic therapy.

9. The system of claim 1, wherein the processing circuitry is further configured to:
responsive to determining that at least one of the plurality of breach conditions is satisfied:
cause injection of the fluid to be stopped; and
cause suction exerted on the second vacuum line to be stopped.

10. The system of claim 1, wherein the processing circuitry is further configured to:
responsive to determining that at least one of the plurality of breach conditions is satisfied:
generate one or more alerts notifying a user to monitor gas egress using fluoroscopy and/or to be careful when retracting the catheter.

11. A method comprising:
delivering cryogenic therapy to a patient via a system comprising:
a catheter comprising a plurality of balloons that at least comprises an outer balloon and an inner balloon positioned within the outer balloon;
a plurality of sensors, each sensor of the plurality of sensors configured to measure a corresponding parameter of a plurality of parameters; and
a console comprising:
a first vacuum line and a second vacuum line, each fluidly coupling the console and the catheter; and
processing circuitry;
determining, by the processing circuitry, whether at least one breach condition of a plurality of breach conditions is satisfied based on at least one of the plurality of parameters, wherein satisfaction of at least one of the breach conditions indicates breach of the plurality of balloons; and
responsive to the processing circuitry determining that at least one of the breach conditions is satisfied:
causing, by the processing circuitry and at a first time, suction to be exerted on the first vacuum line; and
causing, by the processing circuitry and at a second time that is after the first time, suction to be exerted on the first vacuum line and the second vacuum line.

12. The method of claim 11, wherein the console further comprises an injection line, fluidly coupling the console and the catheter, wherein delivering cryogenic therapy to the patient via the system comprises injecting a fluid from the console to the catheter via the injection line;
wherein optionally the plurality of parameters comprises an injection line pressure parameter, and wherein the plurality of sensors comprises an injection line pressure sensor, positioned within the console, configured to measure the injection line pressure parameter by measuring a pressure within the injection line.

13. The method of claim 12, wherein determining whether at least one breach condition of the plurality of breach conditions is satisfied based on at least one of the plurality of parameters comprises determining, by the processing circuitry, that a concurrent pressure change breach condition of the plurality of breach conditions is satisfied when, within a predetermined period of time:
a slope of the distal pressure parameter decreases by an amount greater than or equal to a proximal pressure change threshold value; and
a slope of the proximal pressure parameter decreases by an amount greater than or equal to a proximal pressure change threshold value.

14. The method of claim 11, wherein the plurality of parameters comprises a temperature parameter, and wherein the plurality of sensors comprises a temperature sensor, positioned within the inner balloon, configured to measure the temperature parameter by measuring a temperature within the inner balloon;
wherein determining whether at least one breach condition of the plurality of breach conditions is satisfied based on at least one of the plurality of parameters optionally comprises determining, by the processing circuitry, that a first temperature breach condition of the plurality of breach conditions is satisfied when the temperature parameter is greater than or equal to a temperature threshold value.

15. The method of claim 14, wherein determining whether at least one breach condition of the plurality of breach conditions is satisfied based on at least one of the plurality of parameters comprises determining, by the processing circuitry, that a second temperature breach condition of the plurality of breach conditions is satisfied when a slope of the temperature parameter is greater than or equal to a temperature slope threshold value.

16. The method of claim 12, wherein the plurality of parameters comprises a flow parameter, and wherein the plurality of sensors comprises a flow sensor, positioned within the console, configured to measure the flow parameter by measuring a flow rate within the injection line;
wherein determining whether at least one breach condition of the plurality of breach conditions is satisfied based on at least one of the plurality of parameters optionally comprises determining, by the processing circuitry, that a first flow breach condition of the plurality of breach conditions is satisfied when the flow parameter is less than or equal to a flow threshold value;
wherein determining whether at least one breach condition of the plurality of breach conditions is satisfied based on at least one of the plurality of parameters optionally comprises determining, by the processing circuitry, that a second flow breach condition of the plurality of breach conditions is satisfied when a slope of the flow parameter is less than or equal to a flow slope threshold value.

17. The method of claim 11, wherein determining whether at least one breach condition of the plurality of breach conditions is satisfied based on at least one of the plurality of parameters comprises executing, by the processing circuitry, a machine learning algorithm configured to receive the plurality of parameters as input data.)

18. The method of claim 11, wherein the fluid is cryogen.

19. The method of claim 11, further comprising, responsive to the processing circuitry determining that at least one of the plurality of breach conditions is satisfied:
causing, by the processing circuitry, injection of the fluid to be stopped; and
causing, by the processing circuitry, suction exerted on the second vacuum line to be stopped.

20. The method of claim 11, further comprising, responsive to determining that at least one of the plurality of breach conditions is satisfied, generating one or more alerts notifying a user to monitor gas egress using fluoroscopy.
